(19) United States
(12) Patent Application Publication
(10) Pub. No.: US 2002/0058628 A1
(43) Pub. Date: May 16, 2002

(54) Antioxidants and intracellular glutathione raising agents for therapeutic treatments

(76) Inventors: Mark David Noble, Sandy, UT (US); Margot Mayer - Proschel, Sandy, UT (US)

Correspondence Address: KLAUBER & JACKSON, 411 HACKENSACK AVENUE, HACKENSACK, NJ, 07601

(21) Appl. No.: 09/851,717

(22) Filed: May 9, 2001

Related U.S. Application Data
(63) Continuation of application No. 09/094,100, filed on Jun. 9, 1998, now abandoned.

(51) Int. Cl. A61K 38/06; A61K 31/57; A61K 31/355; A61K 31/375

(52) U.S. Cl. 514/18; 514/178; 514/458; 514/475

(57) ABSTRACT

The present invention relates to a method for treating a disease or condition for which treatment involves the promotion of growth of neuronal cell processes and whose therapy comprises the administration of an agonist for neuronal cell surface receptors that promote outgrowth of neuronal processes said method comprising administering a therapeutically effective amount of said agonist in combination with a therapeutically effective concentration of one or more antioxidants/free radical scavengers and/or an agent capable of raising intracellular thiol levels and/or a steroid. A representative intracellular thiol is glutathione, the agent capable of raising intracellular thiol levels is N-acetylcysteine (NAC), and representative antioxidantsfree radical scavengers may be selected from the group consisting of Vitamin C, Vitamin E, analogs thereof and mixtures thereof. A representative Vitamin E analog is trolox. In a particular embodiment, the steroid may be progesterone and the agonist for neuronal cell surface receptors that promote outgrowth of neuronal processes is an extracellular matrix molecule or active fragment thereof.

### FIELD OF THE INVENTION

The invention is directed to therapeutic methods for treating conditions and diseases associated with acute or chronic loss of cells whose therapy comprises the administration of at least one growth factor together with a compound capable of raising intracellular thiol levels and/or one or more antioxidants/free radical scavengers and/or a steroid, for treating injury and disease whose therapy comprises, at least in part, the promotion of outgrowth of neuronal processes through activation of cell surface integrins or members of the immunoglobulin superfamily of cell adhesion molecules. and with improved methods for preventing cell death associated with acute tissue injury through the administration of cocktails that promote cell survival independently of stimulation of cell surface receptors.

### BACKGROUND OF THE INVENTION

The abilities to prevent undesired cell death, and to promote the generation and survival of desired cells, are of considerable importance in present and future medical practice. For example. severe clinical problems are caused by death of oligodendrocytes in multiple sclerosis, death of neurons in diabetic neuropathy and in spinal cord injury and in amyotrophic lateral sclerosis (ALS), death of kidney cells in a variety of kidney diseases, and death of pancreatic islet cells that leads to diabetes. Other examples of deficiencies that relate to the survival and/or the number of a particular kind of cell are the failure of patients to produce sufficient red blood cells, resulting in anemia.

It has become appreciated that two major environmental influences control the generation and survival of all cell types. The first is the presence of toxic compounds. The environment of a cell can induce cell death by the presence of substances injurious to cells. A variety of toxic compounds have been identified, including complement, glutamate, tumor necrosis factor-α (TNF-α), reactive oxidative intermediates (ROIs) and fas ligand, among others. These toxic compounds, and other toxic compounds. have been associated with a large variety of conditions in which cells die and such cell death causes severe clinical consequences. Thus, it is a matter of significant importance to identify therapeutic compounds, or combinations of compounds, that would prevent such cell death and which might be applicable in a clinical setting.

It has been shown that at least some types of cell death caused by exposure to toxic substances can be prevented by the application of growth factors that stimulate receptor tyrosine kinases (RTKs) and receptors associated with tyrosine kinases (RawTKs). For example, application of ciliary neurotrophic factor (CNTF) can interfere with the ability of TINF-α to kill oligodendrocytes (Louis et al., Science 259:689-692 (1993)) and appropriate growth factors also can py (Schubert, D., et al., Proc. Natl. Acad. Sci. U.S.A. 89:8264-8267,(1992); Maiese et al., J. Neurosci. 13:3034-3040, (1993)). The treatment for diseases in which cell death occurs in vivo, however, has been less than satisfactory. Thus, there exists a need to treat disease conditions associated with such forms of cell toxicity more effectively.

The second environmental influence that controls the generation and survival of cells is the presence of growth factors. It is necessary that the environment of cells contains appropriate growth factors, which may include both mitogens and survival factors (or trophic factors), in order that cells may divide and/or survive. Thus far, the known growth factors are proteins that stimulate cell surface receptors, leading to activation of intracellular tyrosine kinases (which may be part of the receptor itself or which may bind to the receptor as a separate molecule). Receptor tyrosine kinases (RTKs) and receptors associated with tyrosine kinases (RawTKs) have been grouped into several classes of related molecules, according to their structural resemblance with one another. In particular, the intracellular domains of these receptors, and the intracellular proteins with which these domains interact to carry out their signaling function, show great resemblance from one receptor to another within a particular family. This means that within a family of growth factors and growth factor receptors, similar rules apply. One example of such a family of molecules is the neurotrophin (TRK) receptors for nerve growth factor (NGF), brain-derived neurotrophic factor (BDNF) and neurotrophin-3 (NT-3), all of which bind to the Trk family of receptors. Other receptor subfamilies include the platelet-derived growth factor receptor subfamily, the fibroblast growth factor subfamily, the insulin receptor subfamily, and others (which are discussed in such reviews as P. van der Geer, T. Hunter and R. A. Lindberg, Ann. Rev. Cell Biol., 1994, 10:251-337. the contents of which are incorporated herein by reference).

It is also known thatan be successfully used to tsufficiency of a particular cell type or types. For example, the growth factor erythropoietin (EPO) is used to treat anemia associated with renal disease or with chemotherapy,and granulocyte-colony stimulating factor (G-CSF) and granulocyte-macrophage colony stimulating factor (GM-CSF) are used to treat neutropenia associated with chemotherapy. Related therapies are being explored or treatment of diseases of the nervous system. For example, clinical trials are currently being conducted with CNTF for the treatment of ALS and with NGF for the treatment of diabetes-associated neuropathy. The therapies, however, administer extremely high levels of the growth factor which, at a minimum, is extremely expensive. Thus, there exists a need to discover adjuvant therapies that would increase the efficacy of growth factor action in promoting cell generation and/or survival and thus decrease the amount of a growth factor needed to yield a desired effect. As one of the major costs of disease treatment is the cost of recombinant growth factors, the discovery of compounds that increase the effectiveness of such factors could significantly reduce the costs associated with administration of the growth factors.

It is also the case that it is desirable to identify means of promoting such critical cellular functions as cell survival without having to utilize agonists for specific receptors. The identification of molecular species. or combinations of molecules, that might allow cell survival to be promoted without having to apply a ligand for a growth factor receptor would be an important step forward in the identification of materials of potential therapeutic usefulness. Such molecules, or combinations of molecules would find ready application in such conditions as spinal cord injury, in which the prevention of cell death following injury would have a major impact on the prospects for recovery of the injured patient.

Along with the soluble growth factors that currently are being used, or considered for use. in therapeutic settings it is also increasingly apparent that cellular adhesion molecules that may have therapeutic applications. Such cellular adhesion molecules consist of two principal families. The first of these are molecules that exert their influence by virtue of being agonists for receptors of the integrin family (e.g., fibronectin, laminin. vitronectin). Native molecules of this class are generally classified as extracellular matrix proteins. These extracellular matrix proteins may be found as secreted molecules in the matrix surrounding cells, but do not have transmembrane domains that make them an integral part of the cell that produces them. Their receptors. in contrast, are integral membrane proteins. The secon adhesion molecules are members of the immunoglobulin superfamily. These molecules, which are integral membrane proteins (or, on rarer occasions, are linked to the cell surface by special lipid-based linkages) are able to bind to other members of this same family on adjacent cells, as well as to other cell surface receptors, including members of the integrin family. For example, the L1 adhesion molecule and the neural cell adhesion molecule (NCAM) are expressed on the surfaces of neurons and glia, and mediate the interactions between neurons that touch each other, and between neurons and glia. L1 on one cell surface binds to L1 on the surface of the adjacent cell in a homophilic interaction, and NCAM exhibits a similar homophilic interaction. In addition, these molecules may also exhibit heterophilic binding interactions with certain other members of the immunoglobulin superfamily.

The proteins of the extracellular matrix, and the members of the immunoglobulin superfamily, play a variety of important biological roles of which two of the most primary ones are the provision of an appropriate substrate for cell migration (which includes axonal growth), and an induction of such migration from a target cell. This occurs in a dose-dependent manner. Both laminin and L1 have been shown to be effective molecules in the promotion of process outgrowth from neurons.

Despite the structural differences between different extracellular matrix molecules, between different cell surface adhesion molecules, and even between the superfamilies to which they belong, all of these molecules appear to share the common property of mediating their effect through signaling pathways inside the cell, such that binding of an agonist molecule at the cell surface triggers biochemical processes within the cell that are in turn the means by which the cell exhibits its appropriate behavior. The dose-dependent manner in which this occurs further indicates the importance of thinking of these molecules as agonists that are binding to and activating particular cellular receptors. Such agonist ligands need not be the native molecule, but may be fragments thereof, antibodies that bind to the requisite receptor so as to change its conformation to an active conformation, or artificial mimetic ligands that also bind to the receptor and cause its activation.

The inventors have unexpectedly discovered that compounds capable of raising intracellular thiol levels, such as N-acetylcysteine (NAC), can be administered to treat diseases or conditions associated with cell death induced by exposure to toxic compounds and/or by failure of the environment to provide trophic factors in sufficient quantities to promote cell survival and in which diseases or conditions the therapy comprises the administration of at least one growth factor. Thus, these compounds can be used to enhance the efficacy of agonist ligands for growth factor receptors.

The inventors have further provided the unexpected discovery that compounds capable of raising intracellular thiol levels, such as NAC, can be administered to treat diseases or conditions in which it would be advantageous to promote outgrowth of neuronal processes and in which such outgrowth is promoted by exposure of neurons to extracellular matrix molecules and/or members of the immunoglobulin superfamily. Thus such molecules can be used to enhance the efficacy of ligand agonists for receptors that promote outgrowth of neuronal processes.

A further unexpected discovery of the inventors is that exposing cells to a combination of compounds capable of raising intracellular thiol levels, such as NAC, in the company of other free radical scavengers, or exposing cells simply to a cocktail of free radical scavengers, enhances still further the ability to treat (i) diseases or conditions associated with cell death induced by exposure to toxic compounds and/or by failure of the environment to provide trophic factors in sufficient quantities to promote cell survival and in which diseases or conditions the therapy comprises the administration of at least one growth factor and (ii) diseases or conditions in which it would be advantageous to promote outgrowth of neuronal processes and in which such outgrowth is promoted by exposure of neurons to extracellular matrix molecules and/or members of the immunoglobulin superfamily.

The inventors have also unexpectedly discovered that combinations of small molecules from within the family of antioxidants, or, as they may also be called, free radical scavengers, such as NAC, Vitamin C, or trolox (a vitaminned with each other and/or in combination with the steroid hormone progesterone, will support cell survival in the absence of an exogenous stimulator of RTK or RawTK activity. Thus, these combinations of small molecules form a suitable formulation for use in clinical conditions where it is desirable to prevent cell death, particularly cell death associated with acute tissue injury, but in which a ligand agonist for a growth factor receptor may not be available for clinical use.

The inventors have further discovered that by combining a ligand agonist for a growth factor receptor with a compound capable of raising intracellular thiol levels, one or more antioxidantsfree radical scavengers and/or progesterone, it is possible to achieve still higher degrees of cell survival and enhancement of the activity of said ligand agonist.

### SUMMARY OF THE INVENTION

It is therefore an object of the present invention to provide therapeutic agents to treat diseases associated with cell death caused by toxic compounds and/or by an insufficiency of growth factors.

It is yet a further object of this invention to provide therapeutic agents to treat diseases or conditions characterized by an insufficiency of a particular cell type or types and whose therapy comprises the administration of at least one growth factor to promote the survival of the particular cell type or cell types.

It is yet a further object of this invention to provide therapeutic agents to treat diseases or conditions in which it would be advantageous to promote outgrowth of neuronal processes and in which such outgrowth is promoted by exposure of neurons to extracellular matrix molecules and/or members of the immunoglobulin superfamily.

In another aspect of the invention, there is provided a method for treating a disease or condition characterized by an insufficiency of a particular cell type or types and whose therapy comprises the administration of at least one growth factor to promote the survival of the particular cell type or cell types, said method comprising administering a therapeutically effective amount of said growth factor in combination with an agent capable of raising intracellular thiol levels.

In a further aspect of the invention, there is provided a method for treating a disease or condition characterized by the need to promote axonal outgrowth as part of the disease therapy and whose therapy consists of administering, either as a soluble substance(s) or as a substrate-attached molecule(s), extracellular matrix molecules and/or members of the immunoglobulin superfamily, said method comprising administering a therapeutically effective amount of said extracellular matrix molecules and/or members of the immunoglobulin superfamily in combination with an agent capable of raising intracellular thiol levels.

In yet another aspect of the invention, there is provided a mng a disease or condition characterized by an insufficiency of a particular cell type or types and whose therapy comprises the administration of at least one growth factor to promote the survival of the particular cell type or cell types, said method comprising administering a therapeutically effective amount of said growth factor in combination with an agent capable of raising intracellular thiol levels and/or at least one free radical scavenger/antioxidant.

In a further aspect of the invention, there is provided a method for treating a disease or condition characterized by the need to promote axonal outgrowth as part of the disease therapy and whose therapy consists of administering, either as a soluble substance(s) or as a substrate-attached molecule(s), extracellular matrix molecules and/or members of the immunoglobulin superfamily, said method comprising administering a therapeutically effective amount of said extracellular matrix molecules and/or members of the immunoglobulin superfamily in combination with an agent capable of raising intracellular thiol levels and at least one additional antioxidant/free radical scavenger.

In yet another aspect of the invention there is provided a method for preventing cell death associated with acute tissue injury, the method comprising administering a therapeutically effective amount of a cocktail of antioxidantsfree radical scavengers for which at least one member of the cocktail is an agent capable of raising intracellular thiol levels.

In yet another aspect of the invention there is provided a method for preventing cell death associated with acute tissue injury the method comprising administering a therapeutically effective amount of progesterone in combination with antioxidant/free radical scavenger.

In a further aspect of the invention, there is provided a method for preventing cell death associated with acute tissue injury, the method comprising administeramount of NAC in combination with an antioxidantfree radical scavenger.

Other objects, features and advantages of the present invention will become apparent from the following detailed description considered in conjunction with the following illustrative drawings.

### BRIEF DESCRIPTION OF THE DRAWINGS

FIGS. 1A and 1B show the effects of NAC, CNTF, and IGF-1 on cells that would otherwise undergo apoptosis.

FIG. 2A shows the effects of Vitamin C and CNTF on cells that would otherwise undergo apoptosis.

FIG. 2B shows the effects of trolox and CNTF on cells that would otherwise undergo apoptosis.

FIG. 3A shows the effects of IFN-γ on cells that would otherwise undergo apoptosis.

FIG. 3B shows the effects of co-exposure of [CNTF+IFN-γ], or [IGF-1+IFN-γ] on cells that would undergo apoptosis.

FIG. 3C shows the effects of [NAC+IFN-γ] on cells that would undergo apoptosis.

FIG. 4 shows the effects of NAC and nerve growth factor (NGF) on cells that would otherwise undergo apoptosis.

FIG. 5 shows the effects of progesterone and NAC on cells that would otherwise undergo apoptosis.

FIG. 6 shows the effect of NAC and trolox or Vitamin C on cells that would otherwise undergo apoptosis. p FIG. 7 shows the effects of [CNTF+IGF-1], [NAC+Vitamin C+Trolox], or both groups of factors added together, on cells that would undergo apoptosis due to trophic factor withdrawal.

FIG. 8 shows the effects of [CNTF+IGF-1] or [NAC+Vitamin C+Trolox+progesterone], on the survival of cells that would undergo apoptosis due to exposure to Lavendustin A.

FIG. 9 shows the effects of NAC on cell division induced by PDGF.

FIG. 10 shows the effects of NAC on cell proliferation induced by EGF.

FIG. 11 shows the effects of NAC on neurite outgrowth induced by laminin or L1.

### DETAILED DESCRIPTION OF THE INVENTION

Many critical cellular processes are controlled by the binding of agonist ligands to proteins on the cell surface. This binding reaction causes the activation of signal transduction pathways within the cell, which in turn causes a chain of biochemical alterations to functionally interacting intracellular proteins. The extent to which an agonist for such cell surface proteins is able to modulate important cellular processes is clearly influenced by many factors, only a portion of which are presently understood.

Among the cellular processes that are controlled by the binding of an agonist ligand to a receptor protein on the cell surface are cell division, cell survival and cell migration. Often included within the general category of cell migration, even though it is only a portion of the cell that is moving, is the outgrowth of neuronal processes.

Stimulation of the above cellular processes is of great medical importance in many different conditions. For example, the promotion of cell division is required for wound healing, for cell replacement in conditions of chronic degeneration (as in the loss of beta-islet cells in Type I diabetes) and for replenishment of cells of the haematopoietic system after radiation therapy or chemotherapy for cancer. The promotion of cell survival is of importance following acute injury and also in chronic degenerative disorders. For example, the ability to protect neurons from cell death would be of great benefit in such conditions as spinal cord injury, head trauma, stroke, Parkinson's disease, Huntingdon's disease and Alzheimer's disease. The ability to protect the myelin-forming oligodendrocytes of the central nervous system from cell death is of importance in such conditions as spinal cord injury, head trauma, premature birth and multiple sclerosis. The ability to promote outgrowth of neuronal processes following damage would be of great importance in, for example, such acute conditions as spinal cord injury and head trauma, and in such chronic conditions as Parkinson's disease, Huntingdon's disease and Alzheimer's disease.

The potential value of being able to modulate such processes as cell division, cell survival, and outgrowth of neuronal process has-spurred great interest in the identification of the specific molecular signals involved in such processes. A large enough number of such molecular signals have been identified to allow the recognition of important shared aspects of these different processes.

An important large class of agonist ligands that stimulate cell proliferation and survival are ligands that bind to and activate receptor protein tyrosine kinases (RTKs) and receptors associated with tyrosine kinases (RawTKs). RTKs form a superfamily of molecules which share the common feature of having a common catalytic domain that phosphorylates tyrosine residues in response to receptor activation. RawTKs have no intrinsic tyrosine kinase activity, but are induced by ligand binding to bind to and activate other proteins with such enzymatic activity. An example of RawTKs of particular interest in the present context are those considered to be members of the gp130 family, whose agonist ligands include such proteins as ciliary neurotrophic factor (CNTF) leukemia inhibitory factor (LIF), oncostatin M, interleukin-6 (IL-6) and granulocyte-macrophage colony stimulating factor (GM-CSF). Other RawTKs also, however, exist.

Thus far, there are more than 75 known RTKs and RawTKs, belonging to at least 15 distinct subfamilies. Features and principals of action of many of these receptors have been the subject of frequent reviews in the literature (for example, P. van der Geer, T. Hunter and R. A Lindberg, Ann. Rev. Cell Biol., 1994, 10:251-337, the contents of which are incorporated herein by reference). All known RTKs share a conserved catalytic domain of about 250 amino acids, with sequence identities ranging from 32% to 95%. Throughout the rest of the protein, regions of sequence homology, and expression of such domains as cringle domains, cadherin domains, EGF-like domains, immunoglobulin-like domains, fibronectin III repeats, and other features allow the grouping of these proteins into structurally related subfamilies, members of which generally prove to have closely related functions.

Subfamilies of RTKs are often named after a particular ligand agonist, but include within them receptors for other agonists. For example, the PDGF receptor subfarmily includes receptors for stem cell factor (SCF, or c-kit), colony stimulating factor-1 (CSF, 1, or c-Fms), as well as such receptors as FLT1, Flk1, Flk2 and FLT 4. The fibroblast growth factor (FGE) receptor family contains receptors for a variety of FGFs, and well as the Cek2 receptor. The epidermal growth factor (EGF) receptor family contains the receptors for the heregulins as well as for epidermal growth factor and transforming growth factor-alpha, the nerve growth factor receptor subfamily contains receptors for all of the NGF-related neurotrophins, and so forth.

The structural and functional similarities of the RTK family in general, and within each receptor subfamily in particular, indicate strongly that any general principles found to apply to one member of a subfamily will apply to all members of that subfamily, and any general principle found to apply to multiple subfamilies will apply to all members of the RTK superfamily. Thus, as it has been found by the inventors that similar principles apply to members of the PDGF receptor subfamily, the NGF receptor subfamily, the insulin receptor subfamily and the EGF receptor subfamily of RTKs. it can be reasonably expected that other members of the RTK superfamily will be found to display similar properties, even if they belong to other receptor subfamilies than those discussed specifically in the accompanying examples.

RawTK subfamilies are similar in their action in principle to RTK families, except that the protein that binds agonist ligand needs to interact with at least one more cellular protein in order to cause signal transduction. For example, signal transduction induced by binding of ciliary neurotrophic factor (CNTF) requires binding of the CNTF binding domain to the leukemia inhibitory factor (LIF) protein and the gp 130 protein (T. Kishimoto, T. Taga and S. Akira, Cell 76:252-262, 1994; N. Stahl and G. D. Yancopoulos, Cell 74: 587-590, 1994, the contents of both of which are incorporated herein by reference). These receptors activate signal transduction processes through the JakStat family of kinases, which also are known to be mediators of signalling from the interferon-gamma receptor. As provided in the examples, both CNTF and interferon-gamma have their activities in cell survival enhanced by application of the principles of the instant invention.

The observation that the principles of the instant invention apply to activation of RawTKs of the gp130 family and also to other proteins (interferon gamma) that exert their influence, at least in part, through activation of Jak/Stat signalling pathways, as well as to members of the RTK superfamily, indicate strongly that these are general principles that apply to many growth factors acting through tyrosine kinase signalling, regardless of whether or these factors are specifically discussed in the accompanying examples. For example, based on the generality of these observations, it is expected that similar principles will apply, for example to the receptor for erythropoietin (EPO) and related receptors, as a non-limiting example.

Still another class of cell surface receptors that alter cell behavior as a consequence of the binding of an agonist ligand are the integrins (reviewed, for example, in C. Chothia and E. Y. Jonnes, Ann. Rev. Biochem. 66:823-862, 1997, the contents of which are incorporated herein by reference). Integrins constitute a large, widespread family of cell adhesion molecules implicated in biological functions such as embryogenesis, the immune response, wound healing, inflammation and maintenance of tissue integrity. Integrins bind to components of the extracellular matrix (such as laminin, fibronectin and collagens and vitronectin) and also to other cell adhesion molecules (such as VCAM-1 the ICAMs, and even to members of the immunoglobulin superfamily of cell surface receptors). The integrins are all heterodimeric cell surface glycoproteins comprised of alpha and beta subunits that each span the membrane once, with a large number of potential alpha and beta subunits joining together to create specific receptors.

One of the functions of integrin receptors is to promote reorganization of the cellos cytoskeleton and thus to promote (or allow) cell motility. A particularly dramatic example of the ability of agonist ligands for the integrins to stimulate such migration is seen in the promotion of outgrowth of neuronal processes by such molecules as laminin or active fragments thereof. That laminin is a promoter of neurite outgrowth is well established in the art (e.g., C. A. Bates and R. L. Meyer, Dev. Biol., 181:91-101, 1997; H. Hall, S. Carbonetto and M. Schachner, J. Neurochem. 68:544-553, 1997; T. B. Kuhn, C. V. Williams, P. Dou and S. B. Kater, J. Neurosci., 18:184-194, 1998, the contents of which are incorporated herein by reference). It is also well known in the art that laminin is composed of multiple subunits, and that these subunits are themselves also able to promote neurite outgrowth (e.g., R. Brandenberger, R. A. Kammerer, J. Engel and M. Chiquet, J. Cell Biol. 135, 1583-1592, 1996; H. Colognato, M. MacCarrick, J. J. O-Rear and P. D. Yurchenco, J. Biol. Chem., 272:29330-29336, 1997, the contents of both of which are incorporated herein by reference).

The observations. as provided in the examples, that the principles of the instant invention have been found, surprisingly, to also enhance the promotion of neurite outgrowth by laminin fiber indicate the generality of the principles provided herein.

Still another family of cell surface receptors that is of great importance in the promotion of outgrowth of neuronal processes is referred to as the immunoglobulin superfamily (as reviewed in F. S. Walsh and P. Doherty, Annu. Rev. Cell Dev. Biol. 1997, 13:425-456, the contents of which are incorporated herein by reference). This superfamily includes within it such neurite-outgrowth promoting molecules as NCAM, L1, CHL1, NgCAM, NrCAM, neurofascin, axonin-1 and DCC-related proteins.

Members of the immunoglobulin superfamily have the unusual property of being able to function as both agonist ligand and receptor protein, through a process known as homophilic binding. For example, L1 (and NgCAM) stimulates outgrowth of neuronal processes, at least in part, by binding to L1 protein (or NgCAM) on the neuronal cell surface. The L1 that functions as agonist ligand can be presented as a membrane- or substrate-bound molecule, or as a soluble protein. L1 protein is of particular interest as a potential promoter of outgrowth of neuronal processes during development and during regeneration following acute or chronic neuronal injury. As with other cell signalling molecules discussed within, L1 functioning as receptor can be stimulated by binding not only of L1 but also by binding of bioactive fragments of L1 and by binding of particular antibodies to L1. In addition, the native agonist that induces activation of a member of the immunoglobulin superfamily of cell adhesion molecules may itself be structurally unrelated, as seen for example in the activation of DCC and frazzled by binding of netrins (reviewed in F. S. Walsh and P. Doherty, Annu. Rev. Cell Dev. Biol. 13:425-456, 1997).

Although it is not entirely known how binding of agonist ligand to neuronal cell surface receptors stimulates outgrowth of neuronal processes, it is clear that signals are transduced upon such binding. Binding stimulates calcium fluxes and other metabolic alterations within the cell, activated receptor may interact with downstream signalling proteins (e.g., src family members) that are used also by, for example, receptor tyrosine kinases, and may even interact directly with receptor tyrosine kinases. For example, it has been suggested (F. S. Walsh and P. Doherty, Annu. Rev. Cell Dev. Biol., 13:425-456, 1997) that L1 and NCAM exert their action, at least in part, through binding to and activation of an FGF receptor protein.

Members of the immunoglobulin superfamily exert their effects on cellular processes not only through homophilic binding but also through binding to other members of the superfamily and also to structurally unrelated proteins. For example, it is known that L1 can bind to integrin receptors (e.g., A. M. Montgomery, J. C. Becker, C. H. Siu, V. P. Lemmon, D. A. Cheresh, J. D. Pancook, Z. Zhao and R. A. Reisfeld, J. Cell Biol. 132:475-485, the contents of which are incorporated herein by reference).

The observations, as provided in the examples, that the principles of the instant invention have been found, surprisingly, to also enhance the promotion of neurite outgrowth by L1 further indicate the generality of the principles provided herein.

Activation of receptors can be achieved by binding of a variety of substances. For example, the general importance of receptor oligomerization in signal transduction (reviewed in M. A. Lemmon and J. Schlessinger, Trends in Biochemial Sciences (1994), pp. 459-463, the contents of which are incorporated herein by reference) is consistent with the ability to generate receptor activation through the use of antibodies able to cross-link receptors. It is also possible to generate artificial receptor mimics that exhibit the same activating properties as the native agonist ligand. For example, it has been possible to generate small peptides that activate the receptor for erythropoietin (EPO) and stimulate erythropoiesis (Wrighton N C, Farrell F X, Chang R, Kashyap A K, Barbone F P, Mulcahy L S, Johnson D L, Barrett R W, Jolliffe, L K, Dower W J, Science Jul. 26, 1996; 273(5274):458464. the contents of which are incorporated herein by reference). These agonists are represented by a 14-amino acid disulfide-bonded, cyclic peptide with the minimum consensus sequence YXCXXGPXTWXCXP, where X represents positions allowing occupation by several amino acids. The amino acid sequences of these peptides are not found in the primary sequence of EPO. The signaling pathways activated by these peptides appear to be identical to those induced by the natural ligand. In addition, it is well known in the art that truncated fragments of native agonist ligands also frequently are bioactive. Thus, it is reasonably anticipated that the principles of the instant invention will apply also to such other means of agonistically activating cell surface receptors to achieve desired outcomes.

Finding means of augmenting cellular signalling processes is of obvious medical importance. Substances that enhance the functional efficacy of substances that promote cell division, cell survival and outgrowth of neuronal processes will find important use in many clinical applications, ranging from treatment of chronic degenerative disorders and acute injury. potentially even to treatment of autoimmune disorders. For example, in this latter category, treatment of multiple sclerosis patients during an acute relapse could conceivably reduce the destruction of oligodendrocytes occuring in the lesions of these patients.

While it is obviously desirable to find means of enhancing efficacy of substances that promote cell division, cell survival and outgrowth of neuronal processes, it has not been readily perceived how this important goal might be achieved.

While it is also obviously desirable to find means of promoting cell survival in the absence of agonists for RTK or RawTK pathways, it has not been readily perceived how this important goal might be achieved.

In U.S. patent application Ser. No. 08/270,059, by the inventors of the instant application, the contents of which are incorporated herein by reference, and in M. Mayer and M. Noble, Proc. Natl. Acad. Sci. U.S.A., 91:7496-7500, the contents of which are incorporated herein by reference, the applicants reported the discovery that certain small molecules could greatly and synergistically augment the efficacy of agonist ligands that promoted cell survival and cell division.

In the instant application the inventors have further extended the principles of their previous investigations and have established a surprisingly broad applicability of the principles of their invention and also have obtained evidence that principles of their invention can be extended to the generation. of therapeutic combinations of compounds that raise intracellular thiol levels and/or antioxidantsfree radical scavengers and/or steroids and which can be used to promote cell survival in the absence of stimulators of RTK or RawTK pathways.

Thiol regulation is one of the important pathways modulated by the principles of the instant invention. Glutathione is a tripeptide thiol that is normally found in all animal cells and most plants and bacteria at relatively high (1-10 millimolar) concentrations, and helps to protect cells against oxidative damage that put otherwise be caused by free radicals and reactive oxidative intermediates (ROIs) produced during cell metabolism or as the results of, for example. drug overdose. Glutathione is itself the major scavenger of reactive oxidative intermediates present in all eukaryotic forms of life and is generally required to protect cells against damage by oxidants. Glutathione reduces (and thereby detoxifies) intracellular oxidants and is consumed by his reaction. Glutathione is oxidized to the disulfide linked dimer (GSSG),which is actively pumped out of cells and becomes largely available for reconversion to reduced glutathione. Thus, unless glutathione is resynthesized through other pathways, utilization of this compound is associated with a reduction in the amount of glutathione available.

As well as being able to interact directly with ROIs to eliminate these damaging reactive species, the antioxidant effects of glutathione are also mediated less directly by the role of this compound in maintaining such other antioxidants as ascorbate and vitamin E in reduced form. Thus, pharmaceutical compounds which elevate glutathione levels work, at least in part, through enhancement of the defense mechanisms seemingly utilized to normally protect tissue from ROI mediated damage. In addition, pharmaceutical compounds that lead to increases in levels of reduced thiols other than glutathione are also expected to be of importance in the principles of the instant invention, due to the important interactions between the various reduced thiols in a cell in achieving intracellular redox balance.

Compounds capable of raising intracellular thiol levels are known in the art and include N-acetylcysteine (NAC), L-2-oxothizolidine-4-carboxylic acid (Meister et al., J. Am. Coll. Nutr. 5:137-151, 1986), GSH-esters (M. E. Anderson, et al., Anal. Biochem. 183:16-20, 1989) and glutathione itself (J. Vina, et al., Bri. J. Nutr. 62:683-691, 1989).

A preferred compound of the present invention capable of raising intracellular thiol levels is NAC, an extensively documented compound (e.g., N. Taniguchi et al., Glutathione Centennial: Molecular Properties and Clinical Implications, Academic Press, New York, 1989; A. Meister, et al., J. Am. Coll. Nutr. 5:137-151, 1986; O. I. Aruoma, et al., Free Radical Biol. Med. 6:593-597, 1989; and J. M. Burgunder, et al., Eur. J. Clin. Pharmacol. 36:127-131, 1989, the contents of all of which are incorporated herein by reference). NAC has been routinely used in humans to replenish liver glutathione following acetaminophen overdose (which leads to a fatal depletion of glutathione in the liver) and in treatment of pulmonary disorders (e.g., O. I. Aruoma, et al., Free Radical Biol. Med. 6:593-597, 1989; J. M. Burgunder, et al., Eur. J. Clin. Pharmacol. 36:127-131, 1989; M. J. Smilkstein ete al., N. Engl. J. Med. 319:1557-1562, 1988; M. R. Holdiness, et al., Clin. Pharmacokinet. 20:123-134, 1991; P. Moldeus, et al., Respiration 50, Suppl. 1, pp. 31-42; B. Olsson et al., Eur. J. Clin. Pharmacol. 34:77-82, 1988 and G. P. Ventresca et al., In: Drugs in Bronchial Mycolog., eds. P. C. Braga and L. Allegra, Raven Press, New York, pp. 77-102, 1989, the contents of all of which are incorporated herein by reference). This nontoxic drug enters cells readily and replenishes the intracellular cysteine required to produce glutathione, thus leading to an increase in glutathione levels. NAC also reacts directly with ROls, thus protecting cells against these toxic compounds. This twofold action of NAC places this compound in a wholly different class from other ROI scavengers (also known as antioxidants and as free radical scavengers), which do not enhance cellular production of glutatione.

The inventors have discovered that compounds capable of raising intracellular thiol levels can be administered to enhance treatment of diseases or conditions associated with and/or characterized by an insufficiency of a particular cell type or types and whose therapy comprises the administration of at least one growth factor to promote the survival of the particular cell type or cell types. The inventors have further discovered that enhancement of activity of growth factors can also be achieved through co-administration of antioxidants/free radical scavengers. Moreover, the inventors have discovered that still greater enhancement of the action of agonist ligands for stimulation of the processes of cell survival, cell division and promotion of process outgrowth from neurons can be achieved by combining exposure to an agonist ligand with co-exposure to combinations of a compound capable of raising intracellular thiol levels and/or antioxidants/free radical scavengers and/or steroids.

Evidence is provided that the principles of the present invention apply broadly to many cell-signalling systems, including PDGF receptors, EGF receptors, IGF-I receptors, CNTF receptors, interferon-gamma receptors, integrins and L1. Thus, these principles apply to RTKs, RawTKs, receptors that activate the Jak/Stat pathway of signal transduction molecules, and cell adhesion molecules of the integrin and immunoglobulin superfamilies.

The principles of the instant invention can be applied to enhance treatment of diseases or conditions characterized by an insufficiency of a particular cell type or types and whose therapy comprises the administration of at least one growth factor to promote the division of the particular cell type or cell types.

The principles of the instant invention also can be applied to provide therapeutic agents to treat diseases or conditions wherein it would be beneficial to prevent cell death due to acute injury or chronic degenerative processes.

The principles of the instant invention also provide therapeutic agents to be used in the enhanced treatment of diseases or conditions in which it would be advantageous to promote outgrowth of neuronal processes and in which such outgrowth is promoted by administering, either as a soluble substance(s) or as a substrate-attached molecule(s), extracellular matrix molecules and/or members of the immunoglobulin superfamily.

The inventors have also discovered that by combining compounds that raise intracellular thiol levels with antioxidantsfree radical scavengers and/or steroids it is possible to promote cell survival even in the absence of administration of growth factors, thus providing combinations of small molecules with unique and beneficial effects. Moreover, these combinations of small molecules provide still further enhancement to the action of stimulators of cell survival, cell division and stimulation of outgrowth of neuronal processes.

The principles and compounds presented in the instant invention increase the efficacy of the action of the agonist ligand in promoting cell generation and/or survival, or outgrowth of neuronal processes, and thus decrease the amount of growth factor or cell adhesion receptor agonist required to yield the desired effect, The specific growth factor to be used is decided on a disease-specific basis utilizing knowledge known in the art.

For example, in diabetes patients treated with NGF for diabetic neuropathy, whose patients would also be treated with the compounds of the instant invention. For patients being treated with CNTF, neurotrophins, fibroblast growth factor, IGF-I, etc., for ALS, those patients would also be treated with the compounds of the instant invention to enhance the effectiveness of the applied growth factors. Similarly for Parkinson patients and Alzheimer's patients being treated with neurotrophins, these patients would also be treated with the compound of the instant invention. In a preferred embodiment, the compound that is used to raise intracellular thiol levels is NAC

Additionally, patients with anemia associated with chemotherapy or renal failure are commonly treated with erythropoetin (EPO), and patients with neutropenia are treated with granulocyte-colony stimulating factor (G-CSF) and granulocyte-macrophage colony stimulating factor (GM-CSF). In the aforementioned therapies, the amount of growth factor administered can be decreased by the concomitant administration of a compound that raises intracellular thiol levels, preferably NAC, or by the administration of such combinations of compounds as are described in the instant invention.

The inventors have also discovered that cell death associated with acute tissue injury, such as CNS trauma which includes spinal injury, stroke, surgery, hypoxia, reperfusion injury, kidney failure, acute liver failure, septic shock, infarction, embolism, and necrosis induced by bacteria, viruses or other organisms can be prevented by the administration of combinations of compounds capable of raising intracellular thiol levels and/or antioxidants/free radical scavengers and/or a steroid. Thus, one embodiment of this invention is the use of cocktails of compounds in the prevention of cell death associated with acute tissue injury, in which the compound contains a combination of compounds capable of raising intracellular thiol levels and/or antioxidantsfree radical scavengers and/or a steroid. Preferred embodiments include NAC as a compound to raise intracellular thiol levels, vitamin C, vitamin E, or their analogs (e.g., trolox as an analog for Vitamin E) as antioxidantsfree radical scavengers, and progesterone as a steroid.

The treatment of the patient may be with the compounds of the present invention or their salts. Such salts include salts with pharmacologically acceptable cations including, e.g., alkaline or alkaline-earth metals, specifically sodium, potassium or calcium, or salts with physiologically acceptable bases, e.g., simple amines such as ammonia, and in particular with basic amino acids such as lysine, arginine and the like. Preferred compounds to raise intracellular thiol levels are NAC and its salts.

The compounds of the present invention may be formulated in a variety of ways. These include but are not limited to: solid forms, such as powders, granulates, tablets, capsules, dragees; liquid forms such as sterile injectable solutions, solutions or suspensions for oral administration; suppositories; aerosols; and topical or ingestible slow-release formulations The formulations may include conventional additives, such as favored, excipients, stabilizers, effervescent agents, antioxidants, or the like. These additives will be used in conventional amounts and, with the exception of excipients, will usually be present in a total amount of less than about 10 wt %. For slow release particles, various physiologically acceptable biologically degradable polymers may be employed, such as polylactates, polyglycolates, polyaldehydes, polyanhydrides, and the like.

Liposomes may also be employed as carriers, wherein the compounds of the present invention are present in the lumen of the liposome. Preparation of liposomes is conventional and is extensively described in the literature, and need not be described here. In a preferred embodiment, the concentration of the thiol-raising compound, and such other compounds as may be included, will generally be in the range of about 0.01 mM to about 10 mM. The particle size of the liposomes will generally the in the range of about 1 to 500 microns. A farther improvement in delivery of the therapeutic agent can be achieved, for those diseases where the disease is associated with damage to specific cells, by conjugating to the liposomes molecules which provide for specific targeting. For example, antibodies may be found to the liposome, either covalently or noncovalently, where the antibodies may be specific for oligodendrocytes, neurons, kidney cells, or such other cells as may benefit from such targeted administration of therapeutic compounds.

Any convenient mode of administration of the compounds of the present invention may be employed. Administration may be oral, parenteral, via an enema, topical, or the like, such as by injection, oral tablet or powder solutions or other convenient means. Oral administration is preferred. Administration may be daily, multiple dosages per day, bidaily, or any other convenient period.

The compounds of the present invention are administered in an amount sufficient to treat the disease. An amount adequate to accomplish this is defined as a “therapeutically effective amount” or “efficacious amount”. Amounts effective for this use will depend on the severity of the condition, the general state of the patient, the route of administration, and other factors known to those skilled in the art. For example, the doses of the compound that raises intracellular thiol levels, preferably NAC, could range from 100 mg to 10 grams daily, depending on the severity of disease and specificities of treatment, and whether said compound is administered in combination with a agonist ligand used to promote cell division. cell survival or outgrowth of neuronal processes.

Other objects, features and advantages of the present invention will be apparent to the skilled practicioner of the art by the detailed description of the invention, the accompanying examples and claims. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The following examples provide demonstrations of the efficacy of the instant invention in a number of different instances. These examples are given by way of illustration only, and are not intended to represent in any way limitations on the scope of the invention as it is described elsewhere in this document.

### EXAMPLE 1

Enhancement of cell survival induced by agonists of RJKS and RawTKS, and demonstration of compositions of small molecules that support cells survival in the absence of RTK and RawTK agonists.

I(A) Preparation of Oligodendrocyte Cultures

Purified oligodendrocyte were generated from cultures of 0-2A progenitors derived from 7 day old rats and purified using a specific antibody-capture methodology (Wysocki, L. J., et al., Proc. Natl. Acad. Sci. USA, 75:2844-2848 (1978)) (the contents of which are incorporated herein by reference).

Purified cells were grown on poly-L-lysine coated glass coverslips in Dulbecco's modified Eagle's medium (DMEM) supplemented with 25 μg/ml gentamicin, 2 mM-glutathione, 1 μg/ml bovine pancreas insulin, 100 μg/ml human transferrin, 0.0286% (v/v) BSA pathocyte 0.2 μM-progesterone, 0.10 μM-putrescine, 0.45 4 μM L-thyroxine, 0.224 μM-selenium and 0.49 μM 3,3′,5-triiodo-L thyroxine; modified from Bottenstein and Sata (Bottenstein, J. E., et al., Proc. Natl. Acad. Sci. USA, 765:514-517, (1979) (the contents of which are incorporated herein by reference).

The 0-2A progenitor purification protocol was modified from that previously adapted to the 0-2A lineage (Barres, B. A., et al., Cell, 70:31-46 (1992)) (the contents of which are incorporated herein by reference), using a negative selection with the Ran-2 antibody (Barlett. P. F., et al., Brain Res., 204:339-351 (1981)) to eliminate type-1 astrocytes, followed by anti-GalC antibody (Ranscht, B., et al., i Proc. Natl. Acad. Sci. USA, 79:2709-2713 (1982) treatment to remove oligodendrocytes. The specific antibody phenotype of 0-2A progenitors (A2B5+/GalC) allows purification of these cells from the remaining cells suspension. The suspension was plated on an A2B5 antibody coated dish (Eisenbarth, et al., Proc. Natl. Acad. Sci. USA, 76:4913-4916 (1979) to allow binding of all A2B5+ cells to the plate. The supernatant was removed and the plate was washed with DMEM-BS. Cells were allowed to bind to the specific plates for 20-30 minutes in an incubator at a temperature of 37° C. This procedure yielded 2×10⁵ 0-2A progenitor cells from an initial 2×10⁶ mixed cells culture from a rat optic nerve. The bound cells were removed by gentle scraping and washing, and plated on PLL-coated coverslips in a 24 well plate. After the cells were allowed to adhere for 1 hr, 300 ul of DMEM-BS was added and the cells were grown for 3 days. DMEM-BS induces differentation of all cells into oligodendrocyte (Raff, M. C., et al., Nature, 303:390-396 (1983). By staining parallel coverslips with oligodendrocyte-specific antibodies (see below), it was confirmed that 100% of the cells in such cultures were oligodendrocytes. In the final culture, the number of contaminating A2B5-negative cells (type-1 astrocytes and oligodendrocyte) represented <0.5% of the total cells while 0-2A progenitors were present at <99.5-100%. Antibodies for coating the plates were used at the following concentrations: anti-Ran-2 [2.5 μg/ml], A2B5 antibody [5 μg/ml].

I(B) Rescue of Oligodendrocyte from Apoptosis by CNTF or IGF+NAC

Purified 0-2A progenitors purified according to Example 1(A) were plated at 2,500 cells/coverslips. After 3 days of growth in DMEM-BS, the resulting cultures of pure oligodendrocytes were switched to DMEM alone, a condition that induces apoptosis unless cells are exposed to appropriate growth factors. Cultures of oligodendrocyte received various concentrations of CNTF or IGF-I+/−1 mM NAC. After 3 further days of in vitro growth, cultures were analyzed by standard procedures well known to practitioners of the art.

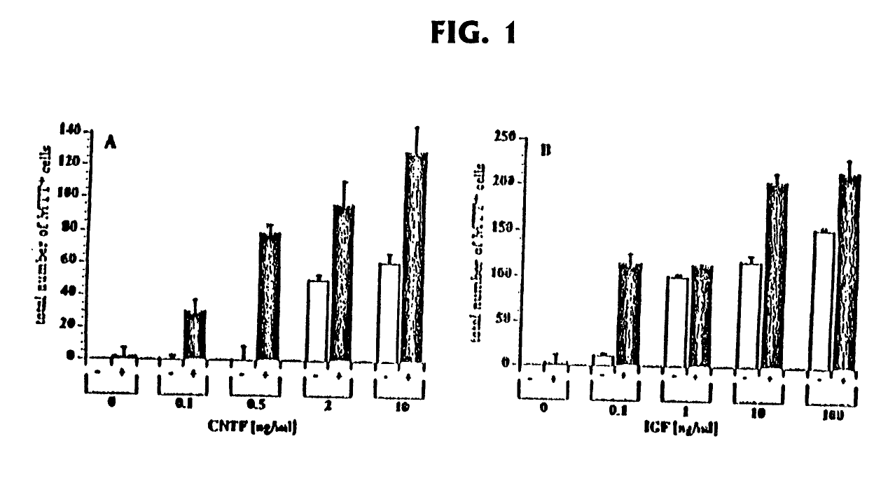

As demonstrated in FIGS. 1(A) and (B), NAC alone did not promote cell survival, but the presence of 1 mM NAC was associated with significant increases in the extent of survival observed with either CNTF or IGF-1 applied by themselves. Values shown are means+/−SD for quadruplicate coverslips from one of four experiments, all of which yielded similar results.

I(C) Rescue of Oligodendrocyte from Apoptosis by CNTF+Vitamin C

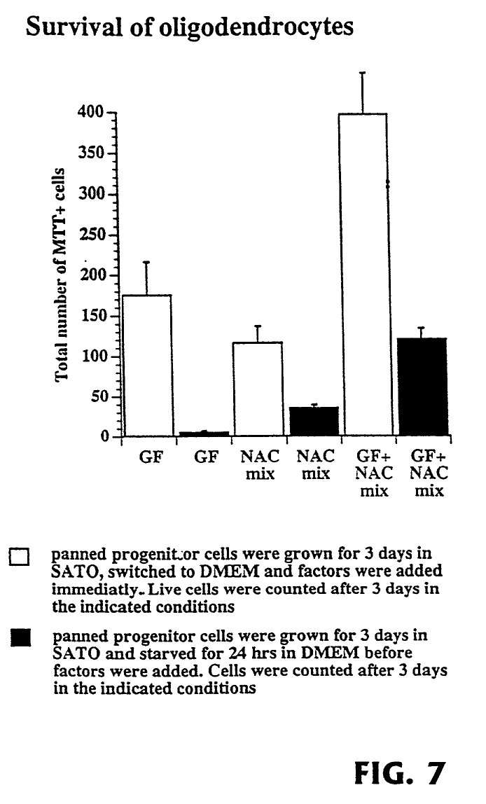

Cells were prepared as in Example (A), except that the initial plating density was 1,000 cells/coverslips. After 3 days in DMEM-BS, the resulting cultures of pure oligdendrocytes were switched to DMEM alone containing 0.5 ng/ml CNTF (a concentration that did not significantly protect cells on its own; see FIG. 6 +1-Vitamin C. After 3 further days of in vitro growth, cultures were analyzed in the same manner as for Example I(B).

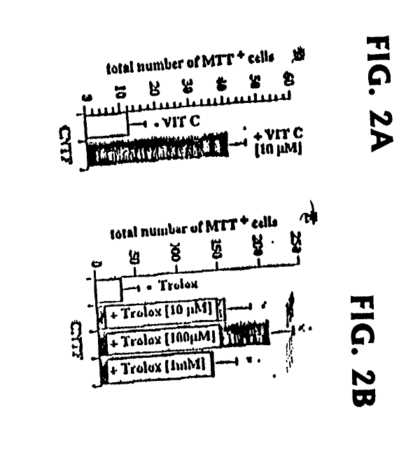

As demonstrated in FIG. 2A, the application of CNTF and Vitamin C together was associated with highly significant (**) increases in the extent of survival observed values shown are means+/−SEM for quadruplicate coverslips from one of two experiments, both of which yielded similar results. The results demonstrate enhancement of the action of a protein trophic factor action by an antioxidant/free radical scavenger that does not act to elevate intracellular glutathione levels. ps I(D) Rescue of Oligodendrocyte from Apoptsis by CNTF+Trolox p Cells were prepared as in Example I(A), except that the initial plating density was 2,000 cellscoverslip. After 3 days in DMEM-BS, the resulting cultures of pure oligodendrocytes were switched to DMEM along to which was added 0.5 ng/ml CNTF+/−Trolox. After 3 further days of in vitro growth, cultures were analyzed in the same manner as experiments examining oligodendrocyte death induced by exposure to toxic stimuli.

As demonstrated in FIG. 2B, the application of CNTF and Trolox together was associated with highly significant (**) increases in the extent of survival observed. Values shown are means+/−SEM for five coverslips from one experiment.

These results demonstrate enhancement of the action of a protein trophic factor by another antioxidant/free radical scavenger that does not act to elevate intracellular glutathione levels.

I(E) Oligodendrocyte Survival Promoted by Interferon-γ is Enhanced by Co-Exposure to NAC but Not by Co-Exposure to Other Growth Factors

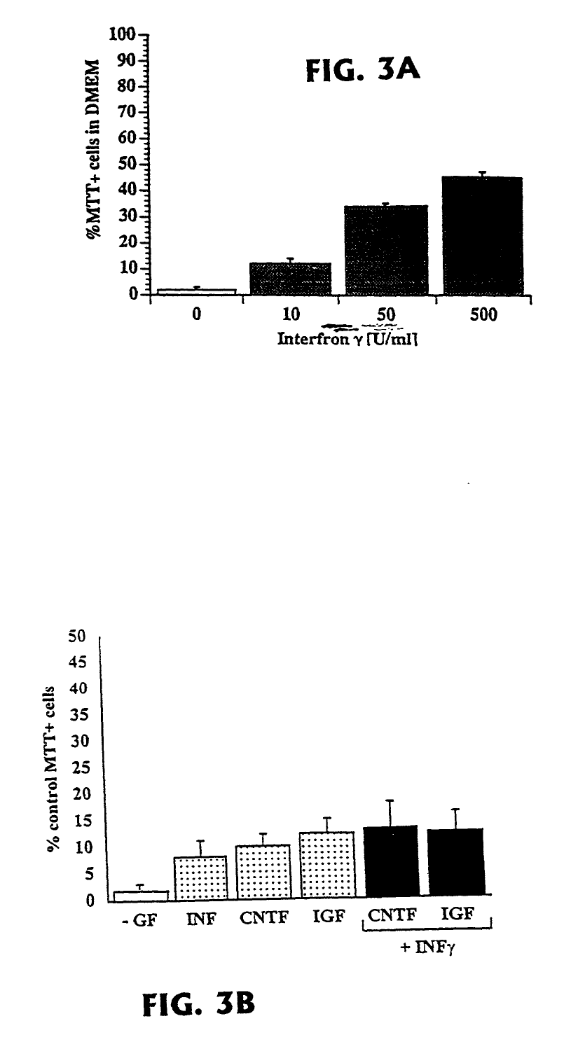

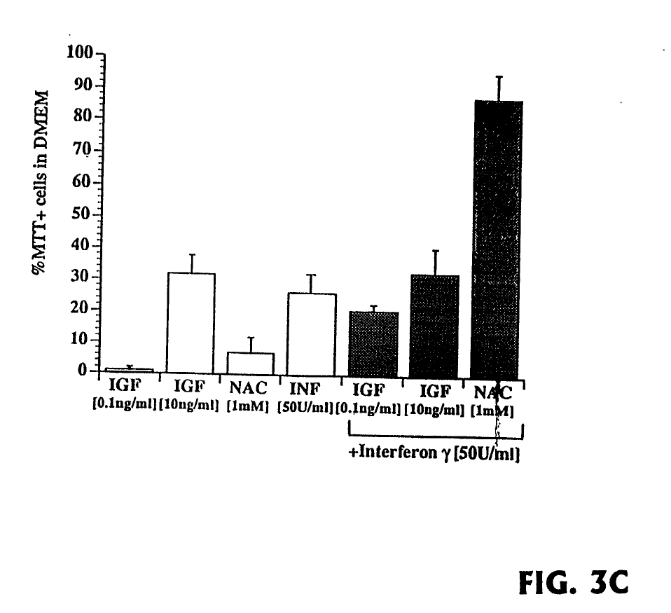

Purified 0-2A progenitor cells were grown on PLL coated glass coverslips at a density of 3,000 cells per coverslip for 3 days to induce differentiation of all cells into oligodendrocytes. Cells were then switched to DMEM alone, supplemented with 10, 50 or 500 u/ml of interferon-γ, and the number of surviving cells present after a further three days was determined by MTT staining. As shown in FIG. 3A, interferon-γ promoted oligodendrocyte survival in a dose-dependent manner.

Growth of oligodendrocytes in the same manner was used to determine whether the survival-promoting activity of interferon-γ was synergistically enhanced by co-exposure to other growth factors (FIG. 3B). Suboptimal concentrations (1 u/ml) of interferon-γ, sufficient to support survival of a mimimally significant number of oligodendrocytes, were combined with equivalently suboptimal concentrations of two other oligodendrocyte survival factors, CNTF (0.5 ng/ml) or IGF-I (0.5 ng/ml). Co-application of IFN-γ+CNTF, or IFN-γ+IGF-I, did not promote survival to any extent greater than any single factor yby itself. A similar lack of synergy was seen at higher concentrations of each growth factor, as seen in FIG. 3B. Here it is illustrated that even at IFN-γ concentrations of 50 u/ml, and IGF-I concentrations of 10 ng/ml, the concentration of IFN-γ plus IGF-I did not promote oligodendrocyte survival to any extent greater than either growth factor applied individually.

In contrast to the lack of enhancement of IFN-γ mediated survival of oligodendrocytes by other growth factors. NAC dramatically enhanced the extent of survival obtained with IFN-γ. As shown in FIG. 3C, NAC (1 mM) by itself did not promote oligodendrocyte survival, but when applied together with IFN-γ dramatically enhanced the extent of survival obtained. As observed in previously discussed examples, this synergy was observed over a range of NAC concentrations and IFN-γ concentrations.

These examples provide yet another example of a separate survival factor whose activity is enhanced by NAC. As seen for other survival factors, the antioxidants/free radical scavengers Vitamin C and trolox had similar, but lesser effects, indicating that other antioxidants are efficacious at enhancing the activity of growth factors.

I(F) Rescue of Embryonic Sensory Neurons from Apoptosis by NGF+NAC

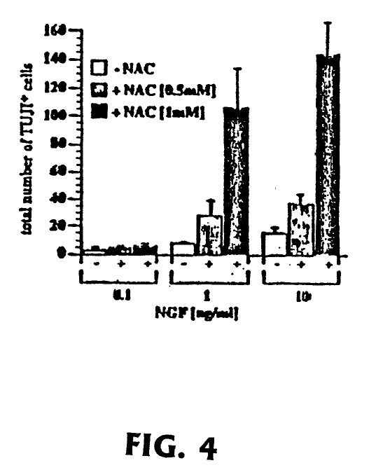

Cultures of embryonic sensory neurons were generated according to methods described in Groves et al., Dev. Biol., 159:87-104, (1993), the contents of which are incorporated herein by reference. The cells were exposed to sub-optimal doses of NGF. As shown in FIG. 4 cultures exposed to NAC and either 1 or 10 ng/ml NGF on days 1 and 2 and labeled on day 3 contained 300-1000% more neurons than those exposed to NGF alone, depending upon the concentrations of NAC and NGF applied.

Neurons were defined by morphology and by labeling with the TUJI antibody against β-tubuli (Groves, A. K., et al., Dev. Biol., 159:87-104 (1993); Moody, S. Q., et al., J. Comp. Neurol., 279:567-580 (1989)) cultures were in DMEM-BS. There was no BrdU labeling of neurons, nor was there any correlation between the extent of BrdU labeling in non-neuronal populations and the number of neurons present (data not shown). Analysis of DNA synthesis by BrdU incorporation (Gatzner, H. G., et al., Science, 318:474-475 (1982) confirmed that this increase in neuronal number was not due to cell division, strongly implicating differential cell death as the reason for the difference.

Values shown are means+/−SD for quadruplicate coverslips from the one of four experiments, all of which yielded similar results.

I(G) Rescue of Oligodendrocyte from Apoptosis by NAC+Progesterone

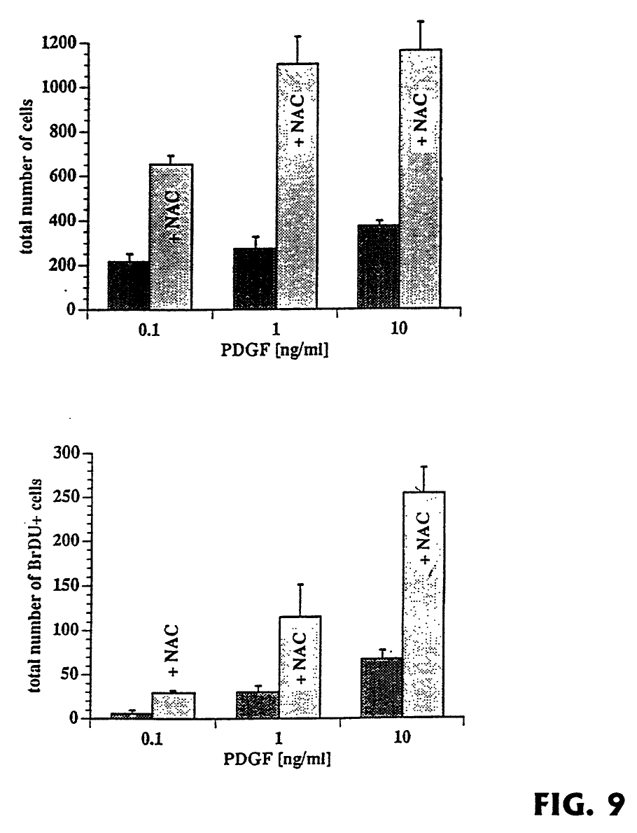

Cells were prepared as Example I(A), except that the initial plating density was 2,500 cell/coverslip. After 3 days in DMEM-BS, the resulting cultures of pure oligodendrocyte were switched to DMEM alone to which was added progesterone+/−1 mM NAC (FIG. 9). After 3 further days in vitro growth, cultures were analyzed as in Example I(A).

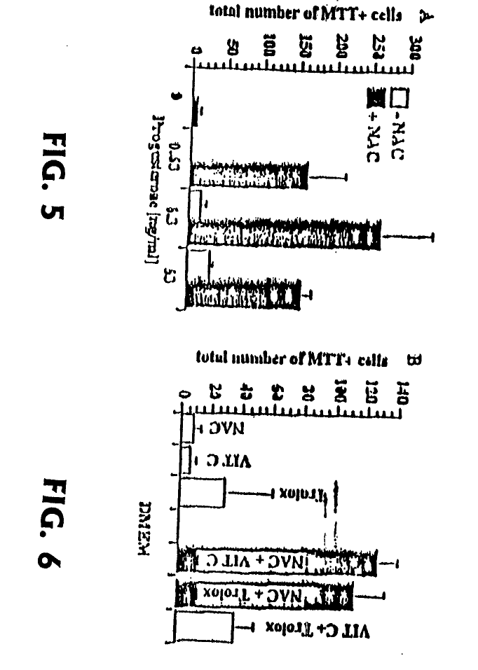

As demonstrated in FIG. 5, the presence of 1 mM NAC in combination with progesterone was associated with highly significant increases (**) in the extent of survival observed. Values shown are means+/−SEM for quadruplicate coverslips from one of two experiments, both of which yielded similar results. These results demonstrate than a compound capable of increasing intracellular glutathione levels can act synergistically with progesterone to promote cell survival even in the absence of agonists of RTKs and/or RawTKs.

I(H) Rescue of Oligodendrocytes from Apoptosis by (a) NAC+Trolox (b) NAC+Vitamin C

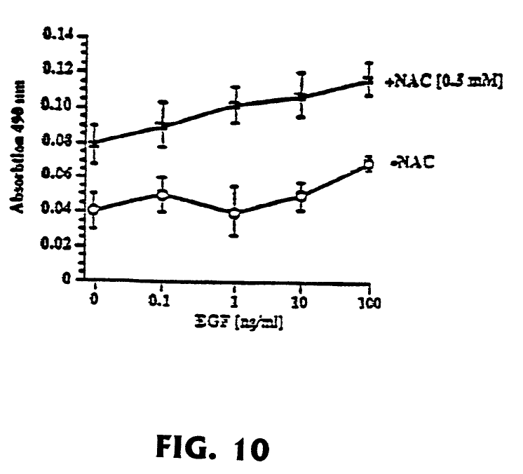

Cells were prepared as in Example I(A), except that the initial plating density ws 2,500 cells/coverslip. After 3 days in DMEM-BS, the resulting cultures of pure oligodendrocytes were switched to DMEM alone to which was added 10 μM Trolox+/−1 mM NAC (FIG. 10). After 3 further days of in vitro growth, cultures were analyzed in the same manner as in Example I(B).

As demonstrated in FIG. 6, no compound alone promoted significant cell survival, nor did the combination of Vitamin C with Trolox, but the presence of 1 mM NAC in combination with either Vitamin C or Trolox was associated with highly significant increases (**) in the extent of survival observed. Values shown are means+/−SEM for quadruplicate coverslips from one three experiments, all of which yielded similar results.

These results demonstrate that a compound capable of raising intracellular glutathione levels can act synergistically with antioxidantsfree radical scavengers to promote cell survival even in the absence of agonists for RTKs and/or RawTKs.

I(I) Rescue of Oligodendrocyte from Apoptosis by (a) Progesterone+NAC+Trolox+Vitamin C, (b) CNTF+IGF-1+NT-3, (c) Progesterone+NAC+Trolox, and (d) Progesterone+NAC+Vitamin C

Cells were prepared as in Example I(A), except that the initial plating density was 2,000 cellscoverslip. After 3 days in DMEM-BS, the resulting cultures of pure oligodendrocyte were switched to DMEM alone to which was added (a) progesterone (63 ug/ml)+NAC (1 mM)+trolox (100 uM)+Vitamin C (10 uM) (“combination”) or (b) CNTF (10 ng/ml)+IGF-1 (50 ng/ml)+NT-3 (5 ng/ml) (“combination”). After 3 further days of in vitro growth, cultures were analyzed as in Example I(B).

In all conditions, the 100% value corresponds to the cell survival seen in cultures of oligodendrocyte not subjected to trophic factor withdrawal.

| | DMEM alone | Combination 1 | Combination 2 |
|---|---|---|---|
| Day 2 | 42 ± 17% | 81 ± 20% | 78 ± 19% |
| Day 3 | 14 ± 3% | 59 ± 10% | 80 ± 20% |
| Day 5 | 0 | 25 ± 8% | 11 ± 3% |

As the data shows, combination 1 was more effective at promoting long term cell survival than combination 2, a combination of RTK and RawTK agonists.

In other experiments. it was determined that Vitamin C can be removed from the combination of progesterone+NAC+Trolox+Vitamin C without loss of activity, and that the combination of progesterone+NAC+Vitamin C is more efficacious than either progesterone+NAC or progesterone+Vitamin C, although, these latter combinations are themselves effective.

I(J) Rescue of Oligodendrocyte from Apoptosis by (a) IFG-1+Vitamin C

Cells were prepared as in Example I(A), except that the initial plating density was 2,000 cells/coverslip. After 3 days in DMEM-BS, the resulting cultures of pure oligodendrocytes were switched to DMEM alone to which was added 0.1 ng/ml of IFG-1. 10 uM Vitamin C, or the combination of 1 ng/ml of IGF-1+10 uM Vitamin C. After 3 further days of in vitro growth, cultures were analyzed in the same manner as in Example I(B).

As shown in the following Table, the combination of Vitamin C with suboptimal doses of IGF-1 was associated with significant levels of oligodendrocyte survival.

| Condition | Survival (as % of DMEM-BS control) |
|---|---|
| IGF-I, 0.1 ng/ml | 1 ± 1 |
| IGF-I, 0.1 ng/ml + 10 uM Vit C | 41 ± 8 |
| 10 uM Vit C | 2 ± 3 |

I(K) Rescue of Oligodendrocyte from Apoptosis by (a) Progesterone+Vitamin C, (b) Progesterone+Trolox

Cells were prepared as in Example I(A), except that the initial plating density was 2,000 cells/coverslip. After 3 days in DMEM-BS, the resulting cultures of pure oligodendrocytes were switched to DMEM alone to which was added progesterone. Trolox, Vitamin C, and NAC, and various combinations thereof. After 3 further days of in vitro growth, cultures were analyzed in the same manner as for Example I(B).

As shown in the Table, the combination of either of these antioxidants with progesterone was associated with significant levels of oligodendrocyte survival, a result not obtained when any of these compounds were used in isolation.

| | | Survival (as % |
|---|---|---|
| Condition | | of DMEM-BS control) |
| Progesterone .63 ng/ml | | 0 |
| Progesterone 6.3 ng/ml | | 4 ± 1 |
| Progesterone .63 ng/ml VitC 10 uM | | 21 ± 6 |
| Progesterone 6.3 ng/ml VitC 10 uM | | 28 ± 4 |
| Progesterone .63 ng/ml Trolox 100 uM | | 27 ± 2 |
| Progesterone 6.3 ng/ml Trolox 100 uM | | 24 ± 9 |
| Trolox | 100 uM | 6 ± 5 |
| VitC | 10 uM | 1 ± 1 |
| NAC | 1 mM | 2 ± 1 |

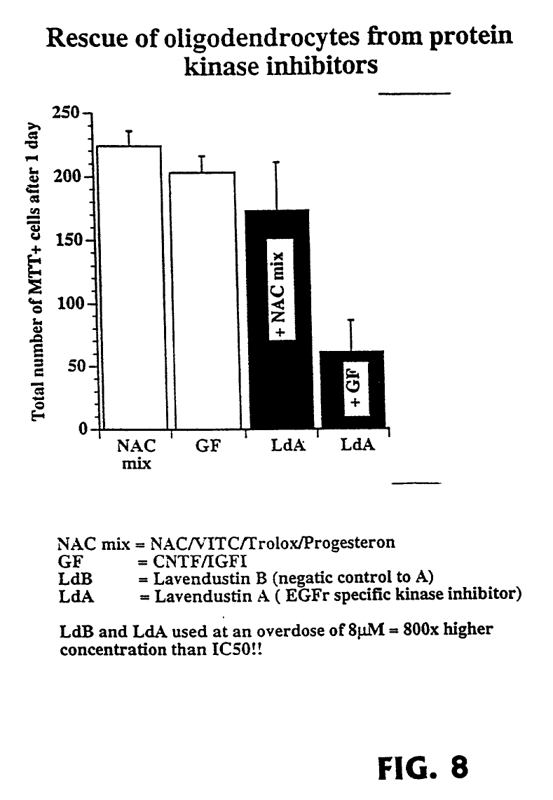

Example I-(L). Survival of oligodendrocytes induced by the combination of NAC, Vitamin C and trolox (“NAC MIX”) is different than for NAC alone, and the combination of NAC mix with a combination of CNTF+IGF-1 is more effective than the NAC mix or the growth factors applied separately (FIG. 8).

Purified oligodendrocytes were generated and grown as in previous examples. 3 days after plating, cells were switched either directly to the indicated conditions (open bars) or were first switched to DMEM alone for 24 hr before the indicated factors were added in an attempt to rescue the cells (black bars). Cells were counted 3 days after switch to the indicated conditions.

As seen previously. NAC, when applied solely by itself was not effective at supporting oligodendrocyte survival. The combination of CNTF+IGF-I did promote survival, but was not able to rescue oligodendrocytes in which cell death was initiated by total survival factor deprivation for 24 hours.

Also as seen previously, the combination of NAC, Vitamin C and trolox did promote cell survival, even in the complete absence of protein trophic factors. A difference in the mode of action of the combination of the glutathione raising agent NAC with other free radical scavengersantioxidants was indicated by the fact that, unlike the combination of CNTF+IGF-I, the NAC Mix was able to rescue a small but significant number of oligodendrocytes in which cell death was initiated by growth for 24 hrs in DMEM alone.

The value of interplay between the action of protein survival factors and the action of NAC mix was demonstrated in two different ways. The extent of oligodendrocyte survival promoted by growth factors plus NAC mix was significantly and synergistically greater than that obtained with either the growth factors or NAC mix applied separately. Moreover, the combination of growth factors plus NAC mix was more effective at rescuing oligodendrocytes in which cell death was initiated by 24 hour growth in DMEM alone.

Example I-M. The combination of NAC, vitamin C, trolox and progesterone (NAC mix) promotes cell survival by a mechanism distinct from that of growth factors, as indicated by the ability of NAC mix but not growth factors to protect against cell death induced by exposure to Lavendustin (FIG. 9) Oligodendrocytes were prepared as in previous examples, and their survival determined after 1 day of growth of cells in the indicated conditions. At this point, the number of oligodendrocytes surviving in the presence of NAC mix or the combination of [CNTF+IGF-I] was identical. If cultures were exposed to Lavendustin A, a protein kinase inhibitor, at a concentration of 8 micromolar, a dramatic and highly significant reduction was seen in the number of oligodendrocytes seen when survival was promoted by growth factors. In contrast, the number of oligodendrocytes present in cultures exposed to NAC mix was not significantly reduced by the exposure to Laverdustin A.

### EXAMPLE II

Enhancement of action of mitogens that promote cell division.

II-A. NAC Enhances the Effect of PDGF on division of Glial Precursor Cells (FIG. 9)

The effect of NAC on generation of 0-2A progenitors and oligodendrocytes was examined in cultures of purified 0-2A progenitors (prepared as in Example IA) exposed to platelet-derived growth factor (PDGF), the best-characterized mitogen for 0-2A progenitor cells. Cells in these cultures were exposed to the indicated amounts of PDGF, and cultures were scored for the total numbers of oligodendrocytes and progenitors (as determined by labeling with anti-GalC and A2B5 antibodies, as in Mayer et al., Glia 8:12-19 (1993). The data demonstrates that the addition of 1 mM NAC to these cultures is associated with a marked increase in the incorporation of bromodeoxyuridine (Brdu) into the nuclei of cells undergoing DNA synthesis, using standard techniques. and for the total number of cells in the cultures. The data demonstrates that the addition of 1 mM NAC to these cultures is associated with a marked increase in the ability of PDGF to induce DNA synthesis and in the total number of cells generated as a consequence of PDGF-induced cell division.

NAC enhances the division-inducing effects of platelet-derived growth factor (PDGF). Purified 0-2A progenitor cells were plated on PLL-coated coverslips at a density of 3,000 cells per cover slip and exposed to PDGF (a known mitogen for these cells) at the indicated concentrations. In addition, cells were exposed to NAC at a variety of concentrations; the effects of 1 mM NAC are illustrated.

In these experiments it was shown that NAC alone had no effect on the induction of cell division (as measured both by preparation of cells engaged in DNA synthesis, as measured by Brdu incorporation, and the total number of cells in the culture). As shown previously, induction of DNA synthesis and increases in cell number by PDGF was dose-dependent.

These experiments revealed the unexpected result that NAC greatly enhanced the responsiveness of cells to stimulation of their division by activation of the PDGF receptor (FIG. 9).

II-B. NAC Enhances the EGF-Induced Proliferation of Embryonic Kidney Cells. (FIG. 10)

FIG. 7 was analyzed as for FIG. 1(B), but used embryonic kidney cells. The cells grown in the 96-well trays were derived by dissociation of kidney rudiments from 11 days embryonic rats. Cells were grown in DMEM-BDS+/−epidermal growth factor (at the concentrations indicated)+/−NAC. The figure demonstrates that at all concentrations of EGF examined, cultures exposed to the EGF+NAC contained more cells than cultures exposed to EGF−NAC. Thus, NAC enhanced the proliferative effects of EGF.

### EXAMPLE III

Enhancement of neurite outgrowth induced by extracellular matrix molecules or by cell surface adhesion molecules. NAC enhances the extent of neurite outgrowth induced by exposure of neurons to laminin or L1.

To determine whether compounds that are able to raise intracellular thiol levels are able to enhance the efficacy of yet a different group of cellular signaling systems, we examined the effects of NAC supplementation on the promotion of neurite outgrowth by laminin and by L1. We first determined suboptimal concentrations for the effect of these cellular adhesion molecules, which were attached to tissue culture plastic surfaces in two different manners. Laminin was directly attached to the tissue culture surface with a cross-linking chemistry that preserves the structure of extracellular matrix molecules. L1 was attached as a dimer chimera, cross-linked by an Fc region from a human immunoglobulin molecule, by first adsorbing to the plastic surface an antibody directed against the human immunoglobulin Fc domain, followed by absorption of the L1 dimer to this surface.

To promote neuronal survival, nerve growth factor (NGF), the survival factor of the spinal ganglion neurons used in this experiment, was also added at different concentrations. As NAC can enhance the effect of NGF on neuronal survival, we utilized several NGF concentrations to ascertain that any effect of NAC did not simply reflect an enhancement of the NGF response of the neurons.

Neurons from spinal ganglia of P7 rats were prepared by dissection of ganglia, dissociation of ganglia with collagenase and trypsin, pre-plating of cell suspensions on tissue culture plastic in the presence of 04 antibody and complement (to remove fibroblasts and to kill Schwann cells), and then plating of the dissociated neurons on the laminin coated or NGF coated surfaces. After 24 hours cultures were stained with an antibody to beta-III-tubulin and neurite length per neuron was measured.

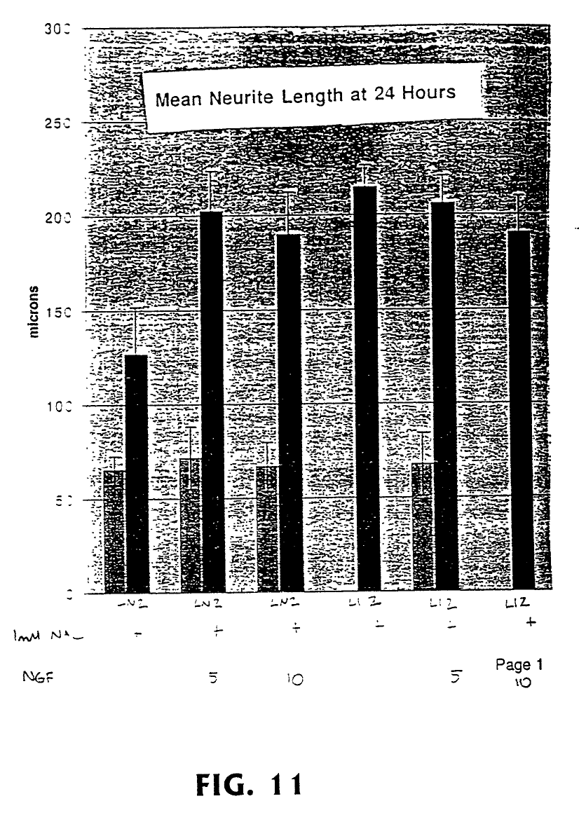

As shown in FIG. 11, neurite length on both laminin (LN) and L1, both present at concentrations of 2 micrograms/ml plating concentration, was enhanced if NAC was present (black bars) as compared with the extent of neurite outgrowth promoted by laminin of L1 in the absence of NAC (grey bars).

It should be understood that the detailed description and the specific examples, while indicating preferred embodiments of the invention, are given by way of illustration only, since various changes and modifications within the spirit and scope of the invention will become apparent to those skilled in the art from this detailed description.

What is claimed is:

1. A method for treating a disease or condition for which treatment involves the promotion of growth of neuronal cell processes and whose therapy comprises the administration of an agonist for neuronal cell surface receptors that promote outgrowth of neuronal processes said method comprising administering a therapeutically effective amount of said agonist in combination with a therapeutically effective concentration of one or more antioxidants/free radical scavengers an0d/or an agent capable of raising intracellular thiol levels and/or a steroid.

2. A method according to claim 1 wherein said intracellular thiol is glutathione.

3. A method according to claim 1 wherein said agent capable of raising intracellular thiol levels is N-acetylcysteine (NAC).

4. A method according to claim 1 where said antioxidants/free radical scavengers are selected from the group consisting of Vitamin C, Vitamin E, analogs thereof and mixtures thereof.

5. A method according to claim 5 wherein said Vitamin E analog is trolox.

6. A method according to claim 1 wherein said steroid is progesterone.

7. A method according to claim 1 wherein said agonist for neuronal cell surface receptors that promote outgrowth of neuronal processes is an extracellular matrix molecule or active fragment thereof.

8. A method according to claim 1 wherein said agonist is for integrins.

9. A method according to claim 1 wherein said agonist for neuronal cell surface receptors that promote outgrowth of neuronal processes is laminin or an active fragment thereof.

10. A method according to claim 1 wherein said agonist for neuronal cell surface receptors that promote outgrowth of neuronal processes binds to a member of the immunoglobulin superfamily.

11. A method according to claim 1 wherein said agonist for neuronal cell surface receptors that promote outgrowth of neuronal processes is itself a member of the immunoglobulin superfamily.

12. A method according to claim 1 wherein said agonist for neuronal cell surface receptors that promote outgrowth of neuronal processes binds to a member of the L1 family of cell adhesion molecules.

13. A method according to claim 1 wherein said agonist for neuronal cell surface receptors that promote outgrowth of neuronal processes is selected from members of the L1 family of cell adhesion molecules, and fragments thereof.

14. A method according to claim 1 wherein said agonist for neuronal cell surface receptors that promote outgrowth of neuronal processes binds to the L1 cell adhesion molecule.

15. A method according to claim 1 wherein said agonist for neuronal cell surface receptors that promote outgrowth of neuronal processes is selected from the cell adhesion molecule L1 and a fragment thereof.

16. A method according to claim 1 wherein said condition is spinal cord injury.

17. A method according to claim 1 wherein said condition is Parkinson's disease.

18. A method according to claim 1 wherein said condition is peripheral nerve injury.

19. A method according to claim 1 wherein said condition is diabetic neuropathy.

20. A method according to claim 1 wherein said condition is chemotherapy-induced neuropathy.

21. A method for treating a disease or condition characterized by an insufficiency of a particular cell type or types said method comprises administration of a therapeutically effective concentration of a combination of one or more antioxidants/free radical scavengers and/or an agent capable of raising intracellular thiol levels and/or a steroid.

22. A method according to claim 21 wherein said intracellular thiol is glutathione.

23. A method according to claim 21 wherein said agent capable of raising intracellular thiol levels is N-acetylcysteine (NAC).

24. A method according to claim 21 where said antioxidants/free radical scavengers are selected from the group consisting of Vitamin C, Vitamin E, their analogs, and mixtures thereof.

25. A method according to claim 24 wherein said Vitamin E analog is trolox.

26. A method according to claim 21. wherein said steroid is progesterone.

27. A method according to claim 21 wherein said condition is spinal cord injury.

28. A method according to claim 21 wherein said condition is head trauma.

29. A method according to claim 21 wherein said condition is stroke.

30. A method for treating a disease or condition characterized by an insufficiency of a particular cell type or types, and whose therapy comprises the administration of an agonist for a receptor protein tyrosine kinase (RPTK) or a receptor associated with a tyrosine kinase (RPTK), said method comprising the administration of said agonist in combination with a therapeutically effective concentration of an agent capable of raising intracellular glutathione levels and/or one or more antioxidants/free radical scavengers and/or a steroid.

31. A method according to claim 30 wherein said intracellular thiol is glutathione.

32. A method according to claim 30 wherein said agent capable of raising intracellular thiol levels is N-acetylcysteine (NAC).

33. A method according to claim 30 wherein said antioxidantsfree radical scavengers are chosen from the group of Vitamin C. Vitamin E, and their analogs.

34. A method according to claim 33 wherein said Vitamin E analog is trolox.

35. A method according to claim 30 wherein said steroid is progesterone.

36. A method according to claim 30 wherein said agonist is an agonist for a member of the platelet derived growth factor (PDGF) receptor subfamily of receptor protein tyrosine kinases.

37. A method according to claim 30 wherein said agonist is an agonist for a PDGF receptor.

38. A method according to claim 30 wherein said agonist is PDGF.

39. A method according to claim 30 wherein said agonist is an agonist for a member of the insulin receptor subfamily of receptor protein tyrosine kinases.

40. A method according to claim 30 wherein said agonist is an agonist for a insulin-like growth factor-I (IGF-I) receptor.

41. A method according to claim 30 wherein said agonist is IGF-1.

42. A method according to claim 30 wherein said agonist is an agonist for a member of the epidermal growth factor (EGF) receptor subfamily of receptor protein tyrosine kinases.

43. A method according to claim 30 wherein said agonist is an agonist for an EGF receptor.

44. A method according to claim 30 wherein said agonist is EGF.

45. A method according to claim 30 wherein said agonist is an agonist for a member of the nerve growth factor (NGF) receptor subfamily of receptor protein tyrosine kinases.

46. A method according to claim 30 wherein said agonist is an agonist for an NGF receptor.

47. A method according to claim 30 wherein said agonist is NGF.

48. A method according to claim 30 wherein said agonist is an agonist for a member of the gp130 subfamily of receptors associated with tyrosine kinases.

49. A method according to claim 30 wherein said agonist is an agonist for a CNTF Receptor.

50. A method according to claim 30 wherein said agonist is CNTF.

51. A method according to claim 30 wherein said agonist is an agonist for a member of the fibroblast growth factor (FGF) receptor subfamily of receptor protein tyrosine kinases.

52. A method according to claim 30 wherein said agonist is an agonist for an FGF receptor.

53. A method according to claim 30 wherein said agonist is a FGF.
